(19) United States
(12) Patent Application Publication
(10) Pub. No.: US 2017/0319048 A1
(43) Pub. Date: Nov. 9, 2017

(54) FLEXIBLE TUBE INSERTION APPARATUS

(71) Applicant: OLYMPUS CORPORATION, Tokyo (JP)

(72) Inventor: Yuichi IKEDA, Tama-shi (JP)

(73) Assignee: OLYMPUS CORPORATION, Tokyo (JP)

(21) Appl. No.: 15/660,084

(22) Filed: Jul. 26, 2017

Related U.S. Application Data
(63) Continuation of application No. PCT/JP2015/052387, filed on Jan. 28, 2015.

(51) Int. Cl. A61B 1/005; A61B 1/00; A61B 1/01; G02B 23/24

(52) U.S. Cl. CPC A61B 1/0055; A61B 1/01; A61B 1/00071; A61B 1/00154; G02B 23/2476

(57) ABSTRACT

A flexible tube insertion apparatus includes a tubular insertion section, a variable stiffness section that causes change of bending stiffness of the insertion section, in regard to continuous segments defined in an axial direction of the insertion section, on a segment-by-segment basis, and a stiffness connecting portion. The stiffness connecting portion is arranged across at least a pair of adjacent segments of the segments. The stiffness connecting portion causes change of bending stiffness of a portion between the pair of segments in such a manner that the bending stiffness of the portion between the pair of segments is continuous with bending stiffness of the pair of segments, in accordance with change of the bending stiffness of the portion between the pair of segments by the variable stiffness section.

### CROSS-REFERENCE TO RELATED APPLICATIONS

This application is a Continuation application of PCT Application No. PCT/JP2015/052387, filed Jan. 28, 2015, the entire contents of all of which are incorporated herein by references.

### BACKGROUND OF THE INVENTION

### 1. Field of the Invention

The present invention relates to a flexible tube insertion apparatus comprising an insertion section to be inserted into a subject.

### 2. Description of the Related Art

It is known that a flexible tube insertion apparatus such as an endoscope apparatus comprising a flexible, elongated insertion section provides improved ease of insertion into a winding part of a subject's body by providing the insertion section with a variable stiffness mechanism. For example, Japanese Patent No. 3752328 discloses an endoscope apparatus comprising an insertion section divided into a plurality of segments as viewed in the longitudinal direction, with a shape-memory alloy actuator arranged in each segment as a variable stiffness mechanism. Each segment is provided with a pressure sensor. In the endoscope apparatus, when the insertion section is inserted into a subject (e.g., the large intestine) and some segment of the insertion section is pressed from outside (e.g., by the intestinal wall), the pressure sensor of that segment senses the pressing and actuates the shape-memory alloy actuator of that segment to decrease the stiffness of that segment.

### BRIEF SUMMARY OF THE INVENTION

One embodiment of the present invention is a flexible tube insertion apparatus comprising a tubular insertion section to be inserted into a subject, a variable stiffness section that causes change of bending stiffness of the insertion section, in regard to a plurality of continuous segments defined in an axial direction of the insertion section, on a segment-by-segment basis, and a stiffness connecting portion that is arranged across at least a pair of adjacent segments of the plurality of segments, and causes change of bending stiffness of a portion between the pair of segments in such a manner that the bending stiffness of the portion between the pair of segments is continuous with bending stiffness of the pair of segments, in accordance with change of the bending stiffness of the portion between the pair of segments by the variable stiffness section.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram schematically showing a configuration of an endoscope apparatus.

FIG. 2 is a block diagram schematically showing a main configuration of the endoscope apparatus.

FIG. 3 is a diagram schematically showing an exemplary configuration of a flexible tube portion.

FIG. 4 is a diagram schematically showing a configuration of a variable stiffness element.

FIG. 5 is a diagram showing a voltage-bending stiffness characteristic of the variable stiffness element.

FIG. 6 is a diagram schematically showing an exemplary configuration of the flexible tube portion according to a first embodiment.

FIG. 7 is a diagram schematically showing another exemplary configuration of the flexible tube portion according to the first embodiment.

FIG. 8 is a diagram schematically showing an exemplary configuration of a flexible tube portion according to a second embodiment.

FIG. 9 is a diagram schematically showing another exemplary configuration of the flexible tube portion according to the second embodiment.

### DETAILED DESCRIPTION OF THE INVENTION

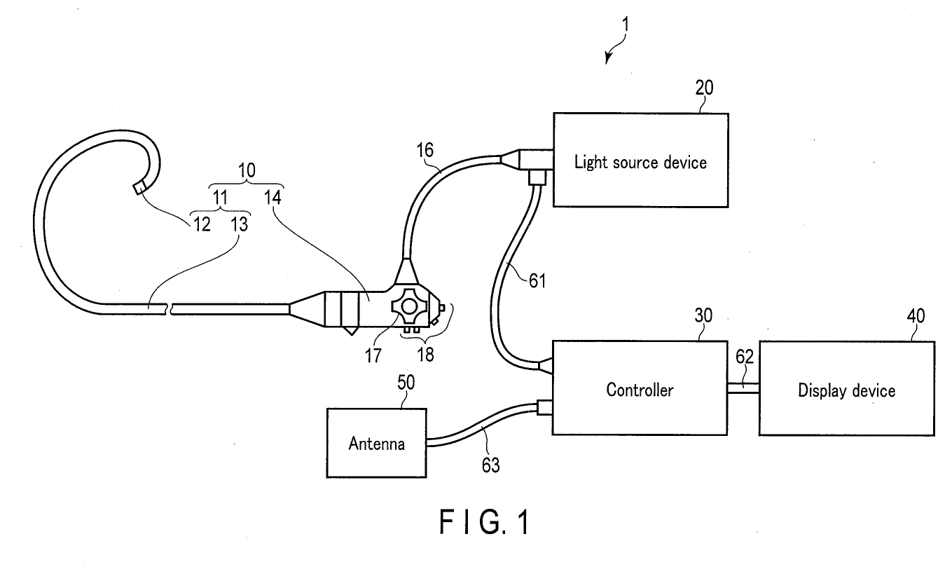

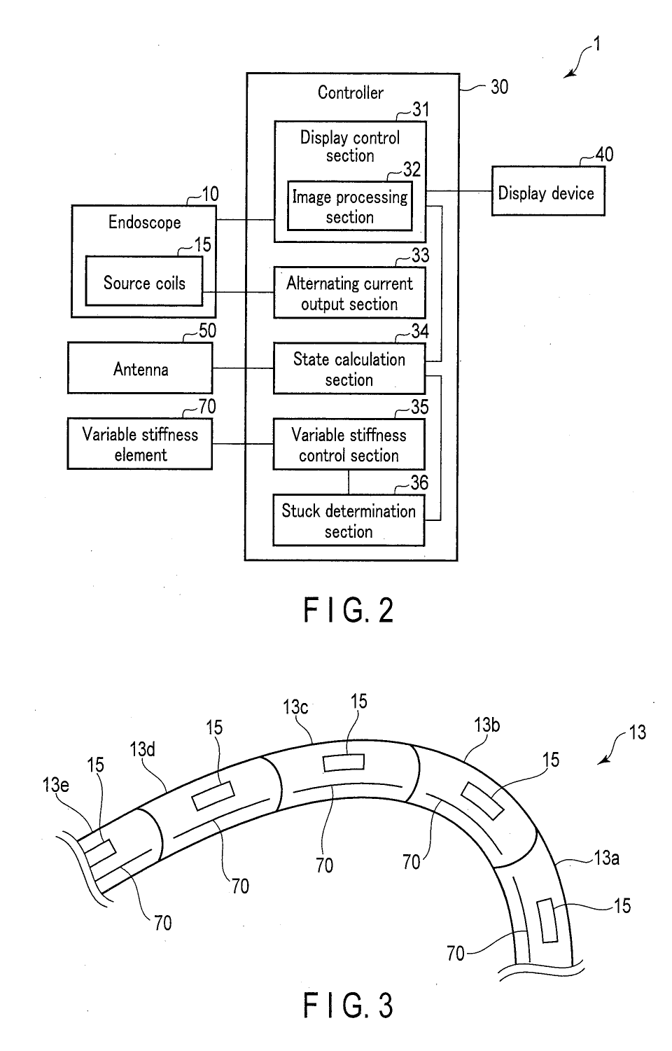

FIG. 1 is a diagram schematically showing a configuration of an endoscope apparatus 1, which is a flexible tube insertion apparatus. FIG. 2 is a block diagram schematically showing a main configuration of the endoscope apparatus 1. The endoscope apparatus 1 comprises an endoscope 10, a light source device 20, a controller 30, a display device 40, and an antenna 50.

The endoscope 10 includes a tubular, elongated insertion section 11 to be inserted into a subject, and an operation section 14 provided on the proximal side of the insertion section 11. The endoscope 10 is, for example, a colonoscope. The insertion section 11 includes a distal rigid portion 12 and a flexible tube portion 13 provided on the proximal side of the distal rigid portion 12. The distal rigid portion 12 incorporates, for example, an illumination optical system (illumination window), an observation optical system (observation window), and an image sensor, not shown in the drawings. The flexible tube portion 13 is a flexible, elongated portion, and comprises a plurality of segments, which will be described later. The flexible tube portion 13 is provided with a plurality of source coils 15 used for detection of the state (bending shape, distortion, etc.) of the flexible tube portion 13 (see FIG. 3). The operation section 14 is provided with, for example, an angle knob 17 and switches 18, used for various operations including a bending operation and an imaging operation of the endoscope 10. A distal end of the flexible tube portion 13 may be bent in any direction by an operation of the angle knob 17.

The light source device 20 is connected to the endoscope 10 via a universal cord 16, which extends from the proximal side of the operation section 14 of the endoscope 10. The universal cord 16 includes, for example, a light guide (optical fiber) connected to the illumination optical system, and an electric cable connected to the image sensor. The light source device 20 supplies light to be emitted from the illumination window of the distal rigid portion 12 via the light guide.

The controller 30 is formed of a device including a processor like CPU or the like, or hardware circuitry like ASIC (Application Specific Integrated circuit), FPGA (Field Programmable Gate Array) or the like. As shown in FIG. 2, the controller 30 includes a display control section 31 (a display control circuit 31) including an image processing section 32 (an image processing circuit 32), an alternating current (AC) signal output section 33 (an AC signal output circuit 33), a state calculation section 34 (a state calculation circuit 34), a variable stiffness control section 35 (a variable stiffness control circuit 35), and a stuck determination section 36 (a stuck determination section 36). The display control section 31 is connected to the electric cable in the universal cord 16 via a cable 61, and thus connected to the endoscope 10 (the image sensor of the distal rigid portion 12). The display control section 31 is also connected to the display device 40 via a cable 62. The AC signal output section 33 is connected to the source coils 15 via a cable, not shown. The state calculation section 34 is connected to the antenna 50 via a cable 63. The variable stiffness control section 35 is connected to a variable stiffness element 70, which will be described later, via a cable, not shown.

The antenna 50 is arranged around the periphery of the subject into which the endoscope 10 is to be inserted. The antenna 50 detects a magnetic field generated by the source coils 15 provided in the flexible tube portion 13. The antenna 50 outputs a detection signal to the controller 30 (state calculation section 34) via the cable 63.

With reference to FIG. 3, the configuration of the flexible tube portion 13 will be further described. FIG. 3 is a diagram schematically showing an exemplary configuration of the flexible tube portion 13. In the flexible tube portion 13, a plurality of source coils 15 are spaced in the longitudinal direction (axial direction) of the insertion section 11, as magnetic field generating elements that generate a magnetic field. The source coils 15 are formed by winding a conducting wire around a magnetic body formed of ferrite, permalloy, or the like. For convenience, let us assume that the flexible tube portion 13 comprises a plurality of segments (virtual units into which the flexible tube portion 13 is evenly divided as viewed in the longitudinal direction) defined in the axial direction thereof. In the example of FIG. 3, five segments 13a, 13b, 13c, 13d, and 13e of the flexible tube portion 13 are shown, and the source coil 15 is arranged in each segment. The source coil 15 provided in each segment is arranged so as to allow the antenna 50 and the controller 30 (state calculation section 34) to detect the state of each segment based on the generated magnetic field. The arrangement of the source coil 15 is not limited thereto, and the source coil 15 may be arranged in only some of the segments.

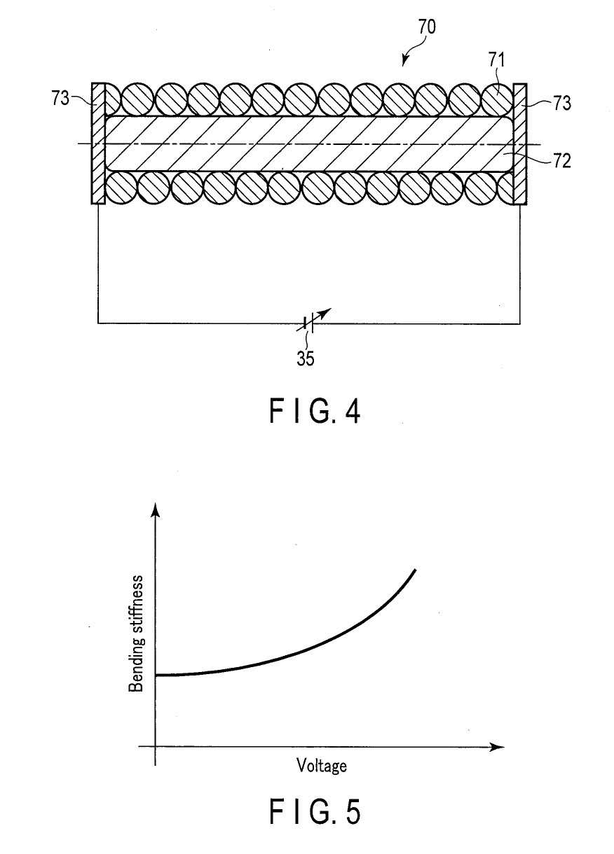

A plurality of variable stiffness elements (variable stiffness actuators) 70 are provided in the flexible tube portion 13. The variable stiffness elements 70 are variable stiffness sections that change the bending stiffness of the flexible tube portion 13 on a segment-by-segment basis in regard to the segments in which the variable stiffness elements 70 are provided. FIG. 4 is a diagram schematically showing a configuration of the variable stiffness element 70. The variable stiffness element 70 includes a coil pipe 71 formed of a metal wire, an electroactive polymer artificial muscle (EPAM) 72 sealed in the coil pipe 71, and electrodes 73 provided on both ends of the coil pipe 71. As shown in FIG. 2, the variable stiffness element 70 is connected to the variable stiffness control section 35, and a voltage may be applied from the variable stiffness control section 35 to the EPAM 72 in the coil pipe 71 via the electrodes 73. The EPAM 72 is an actuator that changes its stiffness and extends and contracts when a voltage is applied. The variable stiffness element 70 is incorporated into the flexible tube portion 13 in such a manner that the central axis of the coil pipe 71 coincides with or is parallel to the central axis of the flexible tube portion 13. The variable stiffness element 70 (EPAM 72) has a stiffness greater than the stiffness of the members forming the flexible tube portion 13.

The electrode 73 (EPAM 72) of the variable stiffness element 70 is applied with a voltage from the variable stiffness control section 35 via a cable, not shown. When such a voltage is applied, the EPAM 73 tends to expand its diameter with the central axis of the coil pipe 71 at its center. However, the EPAM 73 is surrounded by the coil pipe 71, and is restrained from expanding its diameter. Accordingly, the bending stiffness of the variable stiffness element 70 increases as the value of the applied voltage increases, as shown in FIG. 5. That is, when the stiffness of the variable stiffness element 70 is changed, the bending stiffness of the flexible tube portion 13 incorporating the variable stiffness element 70 also changes.

Next, the operation of the endoscope apparatus 1 will be described.

The insertion section 11 of the endoscope 10 is inserted by a user into a subject (from the anus through the rectum into the colon (intestinal tract)). In this case, the insertion section 11 passes through the subject while bending to follow the shape inside of the subject's body. The display control section 31 of the controller 30 obtains an imaging signal output from the image sensor of the distal rigid portion 12 of the insertion section 11 via the electric cable or the like of the universal cord 16. The display control section 31 causes the image processing section 32 to generate an image of the interior of the subject based on the obtained imaging signal. The display control section 31 controls the operation of the display device 40 via the cable 62, and causes the display device 40 to display the generated image.

During insertion, the AC signal output section 33 sequentially applies an AC signal to the source coils 15 via the cable 61 or the like. Each of the source coils 15 generates a magnetic field around the periphery thereof. That is, information regarding the position of the source coil 15 is output from the source coil 15. The antenna 50 detects the position of the source coil 15 based on the output of the source coil 15, and outputs a detection signal to the state calculation section 34. The state calculation section 34 estimates a state (e.g., a three-dimensional shape) of the flexible tube portion 13 (insertion section 11) based on the detection signal from the antenna 50. The information on the estimated state is transmitted to the display control section 31, and a computer graphics image corresponding to the estimated state is generated. The display control section 31 causes the display device 40 to display the generated image. The state calculation section 34 calculates a state quantity (e.g., a bending angle of each segment) indicating the state of each segment, based on the estimated state of the flexible tube portion 13.

The stuck determination section 36 obtains the state quantity of each segment calculated by the state calculation section 34. The stuck determination section 36 determines, from the obtained state quantity, whether or not each segment is stuck (i.e., whether smooth insertion (passage) of the flexible tube portion 13 is prevented by the segment being bent into a V shape). If any of the segments are determined to be stuck, a control signal is transmitted from the stuck determination section 36 to the variable stiffness control section 35, and the stiffness of the variable stiffness element 70 provided in that segment is decreased. This makes that segment soft, eliminating the V-shaped bend. As a result, further insertion into a deep portion of the large intestine is made easy.

The determination by the stuck determination section 36 may be constantly performed in real time during insertion, or may be manually performed by a user by making an input to an input device, not shown, when the patient feels pain that results from pressure on the intestinal wall during insertion.

The stuck determination section 36 may determine whether any of the segments is substantially linear from the obtained state quantity, and transmit, to the variable stiffness control section 35, a control signal to increase the stiffness of the variable stiffness element 70 provided in the substantially linear segment. This prevents the flexible tube portion 13 from being bent at the substantially linear portion, and striking against the intestinal wall, increasing the ease of insertion.

Thus, according to the endoscope apparatus 1, the variable stiffness elements 70 are driven according to the state of the flexible tube portion 13 in the subject, in such a manner that the bending stiffness of the insertion section 11 (flexible tube portion 13) is changed on a segment-by-segment basis, to allow smooth insertion of the flexible tube portion 13 into a deep portion of the winding large intestine.

### First Embodiment

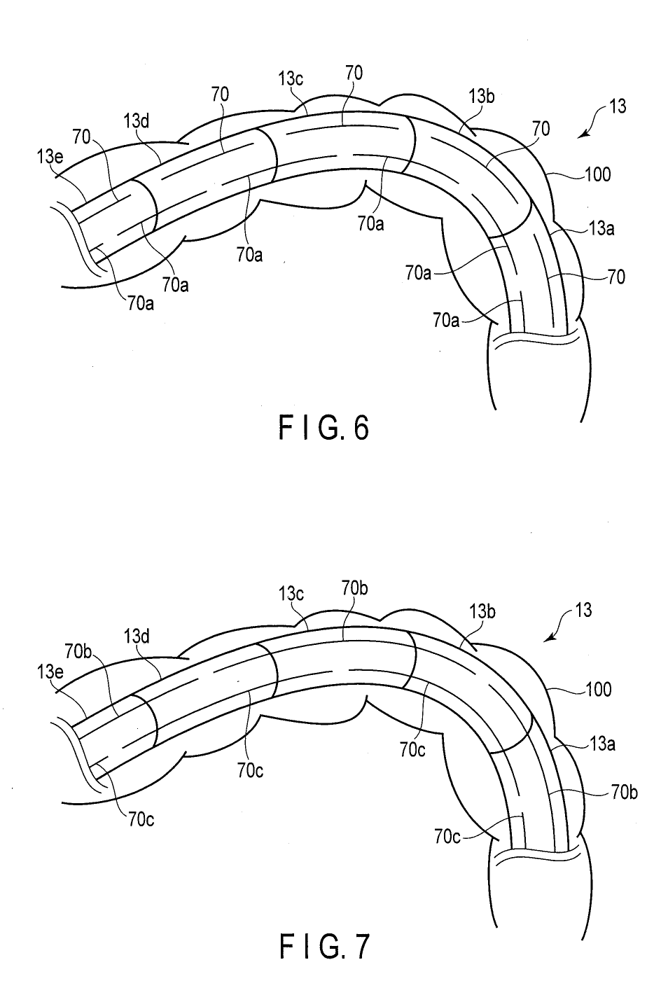

The first embodiment of the present invention will be described with reference to FIGS. 6 and 7.

FIG. 6 is a diagram schematically showing an exemplary configuration of a flexible tube portion 13 according to the first embodiment. In FIG. 6 and the subsequent drawings, the flexible tube portion 13 is shown in a state of being inserted into a large intestine 100, and the source coils 15 are omitted. In FIG. 6, in addition to the variable stiffness element 70 provided in each of the segments 13a, 13b, 13c, 13d, and 13e of the flexible tube portion 13, a variable stiffness element 70a arranged across two continuous (adjacent) segments (for example, segments 13a and 13b, segments 13b and 13c, segments 13c and 13d, and segments 13d and 13e) is provided. The variable stiffness element 70a is a stiffness connecting portion arranged at a connecting portion between the segments, and designed to cause change of bending stiffness of a portion between the segments in such a manner that the bending stiffness of the portion between the segments is continuous with the bending stiffness of the segments (the bending stiffness of the portion between the segments does not abruptly change). The configuration and operating principle of the variable stiffness element 70a are similar to those of the variable stiffness element 70. The length of the variable stiffness element 70 in the longitudinal direction is less than the length of the segment, and the length of the variable stiffness element 70a in the longitudinal direction is greater than a distance between the variable stiffness elements 70 provided in the adjacent segments.

The variable stiffness element 70 is not arranged at the connecting portion (boundary) between the segments, but the variable stiffness element 70a is arranged at least in the connecting portion between the segments. In other words, the variable stiffness elements are arranged without interruption in the longitudinal direction of the flexible tube portion 13. In FIG. 6, for example, the variable stiffness elements 70a are arranged in all areas where the variable stiffness elements 70 are not arranged in the longitudinal direction of the flexible tube portion 13, and are arranged so as to partially overlap the variable stiffness elements 70 in the longitudinal direction.

FIG. 7 is a diagram schematically showing another exemplary configuration of the flexible tube portion 13 according to the first embodiment. In FIG. 7, a variable stiffness element 70b arranged across three continuous segments (for example, segments 13b, 13c, and 13d) and a variable stiffness element 70c arranged across three continuous segments different therefrom (for example, segments 13a, 13b, and 13c, and segments 13c, 13d, and 13e) are provided. The variable stiffness elements 70b and 70c are stiffness connecting portions arranged so as to overlap each other at the connecting portion between the segments, and cause change of the bending stiffness of the portion between the segments in such a manner that the bending stiffness of the portion between the segments is continuous with the bending stiffness of the segments. The configuration and the operating principle of the variable stiffness elements 70b and 70c are similar to those of the variable stiffness elements 70. The variable stiffness elements 70b and 70c are alternately arranged, and are arranged without interruption in the longitudinal direction of the flexible tube portion 13. In FIG. 7, the longitudinal lengths of the variable stiffness elements 70b and 70c are the same, but are not limited thereto. It is only required that the variable stiffness elements 70c are arranged in all areas where the variable stiffness elements 70b are not arranged in the longitudinal direction of the flexible tube portion 13.

For example, when only the variable stiffness elements 70 having a length less than the length of the segment is arranged in each segment, as shown in FIG. 3, the variable stiffness element is absent in the connecting portion between the segments of the insertion section 11. Accordingly, when the variable stiffness control section 35 transmits, to the variable stiffness elements 70 of two adjacent segments, a control signal to increase the stiffness thereof, the connecting portion between the two segments remains soft. Thus, when such a soft connecting portion contacts the intestinal wall or the like and is applied with an external force (reaction), the connecting portion between the segments may become a stiffness-discontinuous portion (a boundary of stiffness occurs) and the flexible tube portion 13 may be bent into a V shape, reducing the ease of insertion. In addition, when the bent flexible tube portion 13 strikes and presses against the intestinal wall, the patient suffers considerable distress.

In the present embodiment, the variable stiffness elements (stiffness connecting portions) are arranged without space even at the connecting portion between the segments in the longitudinal direction of the flexible tube portion 13, and allow the bending stiffness of the flexible tube portion 13 to be adjusted at the boundary between the segments. In the example of FIG. 6, when the stiffness of two adjacent segments is increased, the stiffness of not only the variable stiffness elements 70 of the adjacent two segments, but also that of the variable stiffness element 70a arranged across these segments is similarly increased. Thus, V-shaped bending or buckling does not occur at the boundary between the segments when an external force is applied from the intestinal wall or the like.

Since the variable stiffness elements are thus arranged at the boundary between the segments, a boundary of stiffness does not occur at the connecting portion between the segments, and the bending stiffness is free from discontinuity in the longitudinal direction of the flexible tube portion 13. It is thus possible to provide a flexible tube insertion apparatus that does not cause V-shaped bending at a boundary between the segments when an external force is applied, improving ease of insertion and ease of operation.

Furthermore, according to the present embodiment, during insertion into, for example, the large intestine, it is also possible to reduce the load on the intestinal tract caused by bending of the flexible tube portion 13 at the flexures of the large intestine, including the rectosigmoid flexure, the left colic flexure, and the right colic flexure, thus improving ease of insertion and reducing the patient's distress.

The arrangement and longitudinal length of the variable stiffness elements shown in FIGS. 6 and 7 are shown by way of example, and may be any other arrangement and length if the variable stiffness elements are arranged without interruption in the longitudinal direction of the flexible tube portion 13.

### Second Embodiment

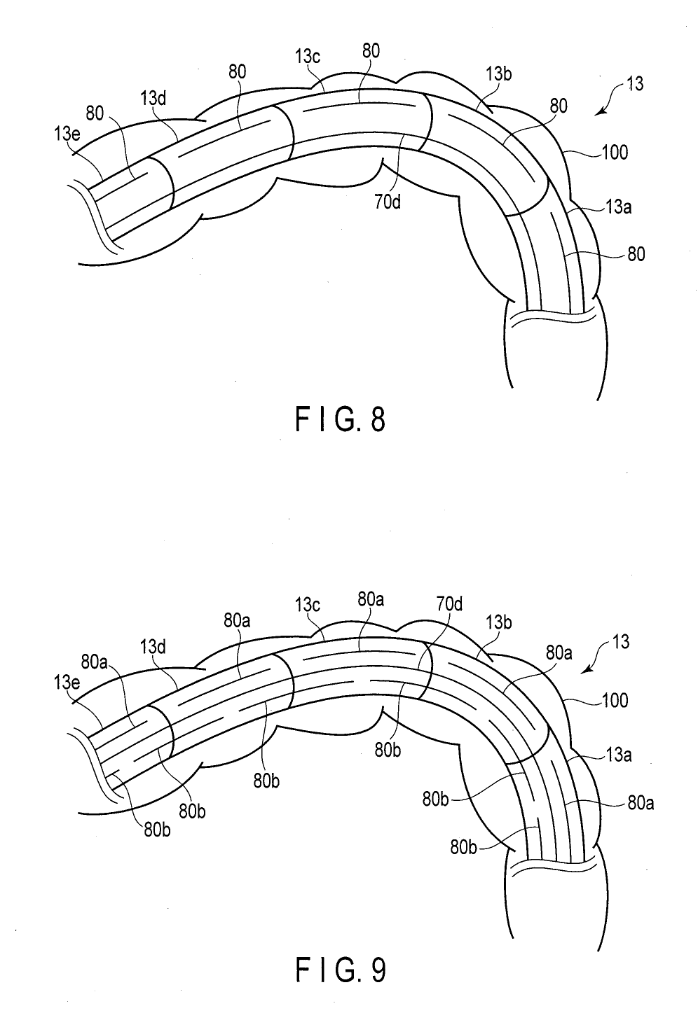

The second embodiment of the present invention will be described with reference to FIGS. 8 and 9.

FIG. 8 is a diagram schematically showing an exemplary configuration of a flexible tube portion 13 according to the second embodiment. In FIG. 8, a long variable stiffness element 70d is arranged as a variable stiffness section in segments 13a, 13b, 13c, 13d, and 13e of the flexible tube portion 13. The configuration of the variable stiffness element 70d is similar to that of the variable stiffness elements 70. The variable stiffness element 70d is one continuous member extending from the segment 13a to the segment 13e, and the variable stiffness element 70 itself also functions as a stiffness connecting portion arranged across a plurality of boundaries between the segments. That is, according to the present embodiment, the variable stiffness element 70d causes change of the bending stiffness of a portion between the segments in such a manner that the bending stiffness of the portion between the segments is continuous with the bending stiffness of the segments.

Each of the segments 13a, 13b, 13c, 13d, and 13e is provided with a voltage application section 80 designed to partially stiffen or soften the variable stiffness element 70d in each segment. The voltage application section 80 is connected to a controller 30 via a cable, not shown, and functions as a variable stiffness control section that causes the stiffness of a portion of the variable stiffness element 70d corresponding to the segment of the voltage application section 80 to be changed, based on a control signal from the controller 30.

When a voltage is applied from the voltage application section 80, the bending stiffness of the variable stiffness element 70d is changed in the segment corresponding to the voltage application section 80. Since the variable stiffness element 70d is a continuous member extending over a plurality of segments, even when the stiffness of a portion of the variable stiffness element 70d is changed by a voltage applied from the voltage application section 80 of one of the segments, the bending stiffness at the boundary between the segments remains continuous, and is free from discontinuity.

FIG. 9 is a diagram schematically showing another exemplary configuration of the flexible tube portion 13 according to the second embodiment. In FIG. 9, a variable stiffness element 70d, which is a long continuous member similar to that of FIG. 8, a voltage application section 80a arranged in each of segments 13a, 13b, 13c, 13d, and 13e, and a voltage application section 80b arranged across two continuous (adjacent) segments (for example, segments 13a and 13b, segments 13b and 13c, segments 13c and 13d, and segments 13d and 13e) are provided. The configuration and the operating principle of the voltage application sections 80a and 80b are similar to those of the voltage application section 80. In FIG. 9, the voltage application sections 80a and 80b are arranged without interruption in the longitudinal direction of the flexible tube portion 13. The voltage application sections 80a and 80b may thus be arranged so as to overlap one another in the longitudinal direction. With such an arrangement, the voltage application sections 80a and 80b are provided without interruption in the longitudinal direction, and the bending stiffness is free from discontinuity.

In the present embodiment as well as the first embodiment, it is possible to provide a flexible tube insertion apparatus that eliminates occurrences of V-shaped bending or buckling at a boundary between the segments when an external force is applied, thus improving ease of insertion and ease of operation.

The arrangement of the variable stiffness elements and the voltage application sections shown in FIGS. 8 and 9 are shown by way of example, and may be any other arrangement, if the stiffness of the variable stiffness elements is continuous in the longitudinal direction of the flexible tube portion 13.

Instead of the variable stiffness element 70d, a shape-memory alloy (hyperelastic alloy) variable stiffness actuator may be used. In this case, the bending stiffness of the shape-memory alloy (hyperelastic alloy) variable stiffness actuator is changed by heating with a heater, instead of providing the voltage application section 80.

While the embodiments of the present invention have been described in terms of the endoscope apparatus 1 comprising the medical endoscope 10, the present invention is not limited to an endoscope apparatus, and includes any flexible tube insertion apparatus comprising a flexible insertion section.

Furthermore, a variable stiffness element different from a variable stiffness element that functions as a variable stiffness section (FIGS. 6 and 7), and a variable stiffness element that functions both as a variable stiffness section and a stiffness connecting portion (FIGS. 8 and 9) have been mentioned as examples of the stiffness connecting portion, but a member having a stiffness greater than the stiffness of the members forming the flexible tube portion 13 may also be used as the stiffness connecting portion.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube insertion apparatus, comprising:
a tubular insertion section to be inserted into a subject;
a variable stiffness section that causes change of bending stiffness of the insertion section, in regard to a plurality of continuous segments defined in an axial direction of the insertion section, on a segment-by-segment basis; and
a stiffness connecting portion that is arranged across at least a pair of adjacent segments of the plurality of segments, and causes change of bending stiffness of a portion between the pair of segments in such a manner that the bending stiffness of the portion between the pair of segments is continuous with bending stiffness of the pair of segments, in accordance with change of the bending stiffness of the portion between the pair of segments by the variable stiffness section.

2. The flexible tube insertion apparatus according to claim 1, wherein the stiffness connecting portion is a member having a stiffness greater than the stiffness of the insertion section.

3. The flexible tube insertion apparatus according to claim 2, wherein the stiffness connecting portion is a member forming the variable stiffness section.

4. The flexible tube insertion apparatus according to claim 3, wherein
the variable stiffness section includes a plurality of variable stiffness elements, and
the stiffness connecting portion is configured in such a manner that two of the variable stiffness elements are arranged so as to overlap one another at a boundary between the pair of segments.

5. The flexible tube insertion apparatus according to claim 3, wherein
the variable stiffness section is a variable stiffness element that continuously causes change of stiffness of two or more adjacent segments of the plurality of segments, and
the stiffness connecting portion is configured in such a manner that the variable stiffness element is arranged across a plurality of boundaries between the segments.
